(19) United States
(12) Patent Application Publication
(10) Pub. No.: US 2006/0234314 A1
(43) Pub. Date: Oct. 19, 2006

(54) Selective binding and analysis of macromolecules

(76) Inventor: Francesco Lorenzo Brancia, Manchester (GB)

Correspondence Address: JACOBSON HOLMAN PLLC, 400 SEVENTH STREET N.W., SUITE 600, WASHINGTON, DC, 20004, US

(21) Appl. No.: 10/516,088

(22) PCT Filed: Jun. 2, 2003
(86) PCT No.: PCT/GB03/02420

§ 371 Date: Sep. 16, 2005

(30) Foreign Application Priority Data
May 31, 2002 (GB) 0212522.7

(51) Int. Cl. G01N 33/53; G06F 19/00; C07D 498/02

(52) U.S. Cl. 435/7.5; 436/518; 702/19; 436/86; 548/304.1

(57) ABSTRACT

There is disclosed a method for selectively binding micromolecules having a lysine functionality comprising the steps of: providing a sample containing one or more species of macromolecules, each having a lysine functionality; providing a binding reagent having the formula X—NH—C(═NH)—OR or X—L—NH—C(═NH)—OR where X is an affinity label that selectively binds to a capture reagent, R is a residue group, and L is a linker moiety; introducing the binding reagent to the sample so as to effect a guanidination reaction between the binding reagent and said one or more species of macromolecules, thereby producing one or more affinity label containing homoarginine derivatives; optionally modifying the affinity label containing homoarginine derivatives to produce further affinity label containing homoarginine derivatives; and capturing affinity label containing homoarginine derivatives using the capture reagent that selectively binds X.

This invention relates to selective binding and analysis of macromolecules with particular, but by means exclusive, reference to quantitative analysis of proteins or protein function in complex mixtures.

Since the completion of the first eukariote -Saccharomyces cerevisiae-, genomic sequence databases of entire organisms have become easily available. So far, the enormous collection of data has been mostly utilised to facilitate the identification of the proteins expressed in the cell. The entire repertoire of proteins synthetised in a cell is also known as proteome.

In a typical proteomics experiment, isolation of the proteins from the rest of cell extract is followed by fractionation of the proteome so that less complex protein mixtures may be obtained using two dimensional gel electrophoresis (2D-GE) and/or chromatographic methods. Thereafter, the resulting protein mixtures are digested using a proteolytic enzyme (trypsin), generating peptide mixtures which are subsequently analysed by mass spectrometry. Protein characterisation relies on the use of soft ionisation techniques such as matrix assisted laser desorption ionisation (MALDI) mass spectrometry

From the genomic sequences it is possible to generate all theoretical proteins expressed at any time (virtual proteome). However, mass spectrometric analysis (either using peptide mass fingerprinting (PMF) or using tandem mass spectrometry in conjunction with collision induced dissociation (CID)) reveals which gene products are actually being synthesised by a cell under a given set of physiological conditions (real proteome). Crucial to the functional analysis of biological systems is the ability to quantify alterations in protein abundance resulting from internal or external perturbations of the cell. In recent years, many efforts have been devoted to develop chemical strategies for relative quantitative analysis of proteins in complex mixtures.

The common strategy involves derivatisation of each set of proteins/peptides with isotopic variants of the same chemical reagent/label. The samples are then combined and differentiated using mass spectrometry after purification. This approach is based upon the assumption that the two sets of isotopically labelled peptides behave identically throughout sample manipulation and mass spectrometric analysis. Oda et al introduced isotopic labelling during cell growth by allowing one cell state to grow in ¹⁵N media and another in ¹⁴N nitrogen (Oda Y, Nagasu T, Chait B T, Nat Biotechnol 2001 Apr. 19(4):379-82). Comparison between the ion intensities of the peptide incorporating both isotopic variants allows relative quantification between two cell states. The approach is expensive and is not applicable to those types of proteins deriving from cells where introduction of isotopic label is difficult. In addition, the mass difference between peptide ion signals causes variations in the level of ¹⁵N incorporation due to peptide composition. Such methods require an initial protein identification step prior to the quantification.

More recently, an alternative strategy based on the selective incorporation of isotopically label amino acids has been proposed. Martinovic et al (S Martinovic, T D Veenstra, G A Anderson, L Pasa-Tolic and R D Smith, J. Mass Spectrom. 2002 37:99-107) described a method in which proteins were extracted from organisms grown in a minimal medium or a minimal medium to which isotopically labelled leucine (Leu D₁₀) was added. The two protein extracts were mixed and analysed by capillary isoelectric focusing (CIEF) coupled with Fourier transform ion cyclotron resonance mass spectrometry (FTICR). The incorporation of the isotopically labelled species shows no effect on the CIEF separation efficiency of the proteins and both isotopically labelled and unlabelled versions of a specific protein are observed in the same spectrum. The difference in the mass shift between the labelled and unlabelled is utilised to determine the number of Leu residues present in a particular protein. A drawback associated with the technique is the high cost of the instrumentation. Pratt and collaborators (J M Pratt, D H L Robertson, S J Gaskell, I Riba-Garcia, S J Hubbard, K Sidhu, S G Oliver, P Butler, A Hayes, J Petty, R Beynon, proteomics 2002, 2, 157-163) have reported the use of selective incorporation of leucine in conjunction with proteolytic digestion and MALDI analysis. In this approach, the utility of leucine labelling is reported as means of enhancing confidence in protein identification in peptide mass fingerprinting (PMF). Because leucine is an abundant amino acid in many genomes, most peptides will contain at least one leucine residue. Labelling in vivo with the two versions of Leu in each digest fragment renders possible identification of the exact number of amino acid residues within the molecule. The extra information can be exploited during an identification process which is conducted using a database searching engine to reduce the search space. In principle the method is amenable to quantification.

However, regardless of its sensitivity and its selectivity, mass spectrometry is still unable to cope with mixtures containing peptides derived from several proteins. The presence of many components generates chemical noise which is detrimental to detection of those ions whose chemical features are relatively poorly suited to the ionisation method employed. Prefractionation is applied in order to resolve a mixture into its components. Of chromatographic techniques, biotin-avidin affinity capture-release based methods followed by liquid chromatography are highly appealing due to the possibility of obviating the use of gel electrophoresis (which is known to be unsuitable in the analysis of low abundance proteins). Of particular relevance to the present application is the isotope coded affinity tag (ICAT) technique. In this approach, proteins extracted from cells can be labelled, separated and quantified (Gygi S P, Rist B, Gerber S A, Turecek F, Gelb M H, Aebersold R, Nat Biotechnol 1999 Oct. 17(10):994-9 and International Publication WO 00/11208, the contents of both of which are hereby incorporated by reference).

The label is composed of three parts: firstly, an affinity tag (typically biotin), which is utilised to isolate ICAT labelled peptides; secondly, a linker incorporating stable isotopes; and thirdly a reactive group with specificity towards thiol groups. The biotin group is selectively recognised during the affinity extract step by an avidin moiety attached to a chromatographic column.

The linker moiety typically contains either eight hydrogens (light) or deuteriums (heavy), and provides for relative quantification. The reactive group having specificity towards thiol groups enables the label to react with cysteine residues. The overall protocol involves derivatisation of all proteins present followed by digestion of the entire set and affinity extraction prior to tandem mass spectrometric analysis. Comparison of the precursor ions of the two isotopically labelled ion species enables the relative abundance of one ion against another to be determined. In a similar approach, Qui and coworkers described a new class of chemically modified resins—termed ALICE—in which a nonbiological polymer with an acid-labile anchor group replaces the biotin moiety (Y. Qui, E. A. Sousa, R. M. Hewick, and J. H. Wang. Acid-labile isotope-coded extractants: a class of reagents for quantitative mass spectrometric analysis of complex protein mixtures 1. Anal. Chem. 74 (19):4969-4979, 2002).

However, there are a number of problems and disadvantages associated with the above described ICAT technique. Firstly, the abundance of cysteine amino acid residues in proteins is relatively low. Secondly, the reactivity of the thiol specific group is not high and, furthermore, side reactions can take place. Thirdly, the ionisation efficiency of the ICAT label is not high. The combined effect of these experimental factors, together with factors such as solvent and electrolyte, is two-fold. Firstly, the sensitivity of the ICAT technique is lower than is desirable. Secondly, the ionisation efficiency is not independent of the protein or peptide being labelled, thereby introducing a substantially unquantified variable into the quantitative analysis.

Other methods targeting different functional groups have been recently reported. Goshe and coworkers presented an approach that utilises phosphoprotein isotope-coded affinity tags (PhIAT) (M. B. Goshe, T. P. Conrads, E. A. Panisko, N. H. Angell, T. D. Veenstra, and R. D. Smith. Phosphoprotein isotope-coded affinity tag approaches for isolating and quantitating phosphopeptides in proteome-wide analyses. Anal. Chem. 73 (11): 2578-2586, 2001). The strategy combines stable isotope and biotin labelling to enrich and quantitatively measure differences in the O-phosphorylation states of proteins.

Although the isotope-coded affinity tag based strategy relies on the affinity chromatography step, which can be easily interfaced with an electrospray instrument, efforts have been made to integrate ICAT with MALDI instruments. Until the peptides are eluted from the microcapillary reversed-phase liquid chromatography column, the procedure is identical to that previously described. After addition of matrix, the sample spots are analysed using a QqTOF mass spectrometer, by first obtaining a mass spectrum of the peptides in each sample spot in order to quantify the ratio of abundance of pairs of isotopically tagged peptides, followed by tandem mass spectrometric analysis to establish the correct sequence of selected peptides for protein identification (T. J. Griffith, S. P. Gygi, B. Rist, R. Aebersold, A. Loboda, A. Jilkine, W. Ens, and K. G. Standing. Quantitative proteomic analysis using a MALDI quadrupole time-of-flight mass spectrometer. Anal. Chem. 73 (5): 978-986, 2001).

The present invention inter alia overcomes the above named problems and disadvantages associated with the ICAT technique.

According to a first aspect of the invention there is provided a method for selectively binding macromolecules having a lysine functionality comprising the steps of:

providing a sample containing one or more species of macromolecules, each having a lysine functionality;

providing a binding reagent having the formula
X—NH—C(═NH)—OR
or
X—L—NH—C(═NH)—OR
where X is an affinity label that selectively binds to a capture reagent, R is a residue group, and L is a linker moiety;

introducing the binding reagent to the sample so as to affect a guanidination reaction between the binding reagent and said one or more species of macromolecules, thereby producing one or more affinity containing homoarginine derivatives;

optionally modifying the affinity label containing homoarginine derivatives to produce further affinity label containing homoarginine derivatives; and

capturing homoarginine derivatives using the capture reagent that selectively binds X.

According to a second aspect of the invention there is provided a method for analysing one or more proteins, protein functions and/or peptides in one or more samples comprising the steps of:

providing a binding reagent having the formula
X—NH—C(═NH)—OR
or
X—L—NH—C(═NH)—OR
where X is an affinity label that selectively binds to a capture reagent, R is a residue group, and L is a linker moiety;

introducing the binding reagent to the one or more samples so as to effect a guanidination reaction between the binding reagent and proteins and/or peptides having a lysine functionality thereby producing one or more affinity label containing homoarginine derivatives;

optionally modifying the affinity label containing homoarginine derivatives to produce further affinity label containing homoarginine derivatives;

capturing affinity label containing homoarginine derivatives using the capture reagent that selectively binds X;

and performing an analysis of affinity label containing homoarginine derivatives.

There are a number of advantages associated with this scheme. The method is specific to lysine amino acid residues, which are more abundant than the cysteine amino acids analysed in Gygi et al, abid. Unlike Gygi et al, the method is not subject to side reactions, since the ε-amino group of the isourea moiety is more specific than the sulphydril moiety of Gygi et al. To date, evidence about conversion of the N-terminus amino groups is very rare, with just a single paper reporting guanidination of the N-terminus when glycine is the N terminal amino acid residue. Furthermore, in comparison to Gygi et al, the binding reagents of the present method provide an increase in ionisation efficiency.

The advantages are also applicable to the first aspect of the invention.

The step of modifying the homoarginine derivatives may comprise converting proteins present into peptides. This conversion might be performed enzymatically or chemically. Alternatively, it is possible to analyse the direct product of the guanidination reaction between the binding reagent and a protein “directly”, i.e. without digestion or fragmentation of the protein into peptides ( top-down approach).

The protein functions and/or peptides may be identified by the analysis of the affinity label containing homoarginine derivatives. The analysis may comprise the step of comparing data generated by an analytical technique with sequence databases. Typically, this is performed using tandem mass spectrometry (MS/MS). It is also possible to interpret data directly.

Relative expression levels of proteins in two or more samples containing proteins may be determined in a method comprising the steps of:

providing a series of binding reagents having the formula
X—NH—C(═NH)—OR
or
X—L—NH—C(═NH)—OR
where X is an affinity label that selectively binds to a capture reagent, R is a residue group, and L is a linker moiety, and wherein the chemical formulae of the binding reagents in the series are identical but each binding reagent in the series comprises a different combination of isotopes so that the binding reagents in the series are isotopically labelled by way of the molecular mass of each binding reagent in the series being different to the molecular masses of the other binding reagents in the series;

introducing a different binding reagent from the series to each sample so as to effect, in each sample, a guanidination reaction between a binding reagent and moieties having a lysine functionality, thereby producing a plurality of isotopically labelled, affinity label containing homoarginine derivatives;

combining the samples;

optionally converting proteins into peptides;

capturing affinity label containing homoarginine derivatives using the capture reagent that selectively binds X; and

performing an analysis of affinity label containing homoarginine derivatives in which the relative abundances of a subset of homoarginine derivatives which differ only by virtue of their isotopic labelling are measured, thereby determining the relative expression levels of the protein from which the subset of homoarginine derivatives originated.

X can be an atom or a functional group, and may be attached to a solid support.

In general, if proteins are to be converted into peptides, then proteins present in the affinity label containing homoarginine derivatives are converted into peptides. However, it is possible to convert proteins in the samples into peptides prior to treatment with binding reagent. In other words, the step of converting proteins into peptides is interchangeable with other steps in the abovedescribed scheme for determining relative expression levels of proteins.

In conjunction with the determination of the relative expression levels of the proteins, the proteins, protein functions and/or peptides may be identified by the analysis of the affinity label containing homoarginine derivatives. The analysis may comprise the step of comparing data generated by an analytical technique with sequence data or of comparing data produced from mixtures differentially derivatised. It is also possible to interpret data directly.

Advantageously, analysis comprises mass spectrometric analysis. Other techniques, such as electron microscopy, might be contemplated. Mass spectrometric analysis may comprise tandem mass spectrometry. This is especially useful in conjunction with peptide/protein sequencing (denovo or database assisted), for identifying proteins, protein function and peptides. One stage of mass spectrometry is conveniently utilised to determine the relative abundances of a subset of homoarginine derivatives which differ only by virtue of their isotopic labelling, through comparison of ion peaks in a mass spectrum associated with each homoarginine derivative in the subset.

Tandem mass spectrometry of product ions differing by a discrete and known value can generate product spectra whose interpretation can be obtained by comparing the ions present within the spectrum. The predictable difference in mass between ions can be utilised to identify ion series and to facilitate interpretation of data in conjunction either with database searching strategy or with the advice of the de-novo sequencing software.

A binding reagent may be utilised in order to capture and isolate proteins and other types of molecules bearing a primary amine from a complex mixture prior to characterisation.

The method may further comprise the step of releasing captured affinity label containing homoarginine derivatives from the capture reagent prior to the step of performing an analysis. The capture reagent may comprise part of a chromatographic separation system which separates chemically different affinity label containing homoarginine derivatives. Conveniently, the chromatographic separation system utilises liquid chromatography. LC-MS systems may be employed. However, other methods can be used, such as other chromatographic separation systems, for example, gas chromatography. The homoarginine derivatives might be separated out in situ through immobilisation on a chip or another solid surface or matrix, in which instance of step of releasing captured affinity label containing homoarginine derivatives is not mandatory.

Absolute quantification of the proteins and/or peptides may be obtained.

In the first and second aspects of the invention, R may be a moiety that is compatible with the guanidination reaction. R may be an alkyl group, such as CH₃, C₂H₅ or C₃H₇. X may be an alkyl group, such as CH₃. The binding reagent may be CH₃CONHC(═NH)OCH₃. L can be an alkyl chain.

According to a third aspect of the invention there is provided a reagent for selectively binding macromolecules having a lysine functionality having the formula
X—NH—C(═NH)—OR
or
X—L—NH—C(═NH)—OR
where X is an affinity label that selectively binds to a capture reagent, R is a residue group, and L is a linker moiety. X may be biotin or a modified biotin, or an alkyl group such as CH₃. R may be a moiety that is compatible with the guanidination reaction. R may be an alkyl group, such as CH₃, C₂H₅ or C₃H₇.

Methods and reagents in accordance with the invention will now be described with reference to the accompanying drawings in which:—

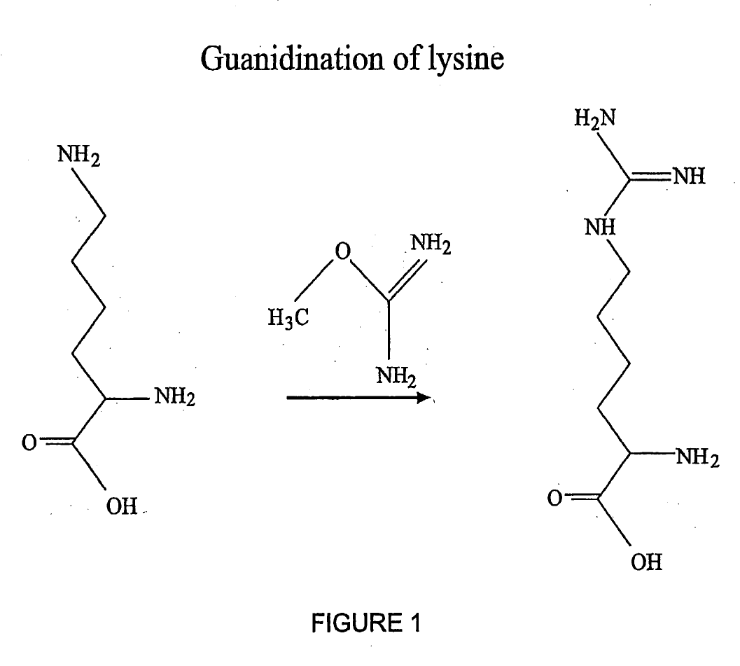

FIG. 1 shows the guanidination of lysine into homoarginine;

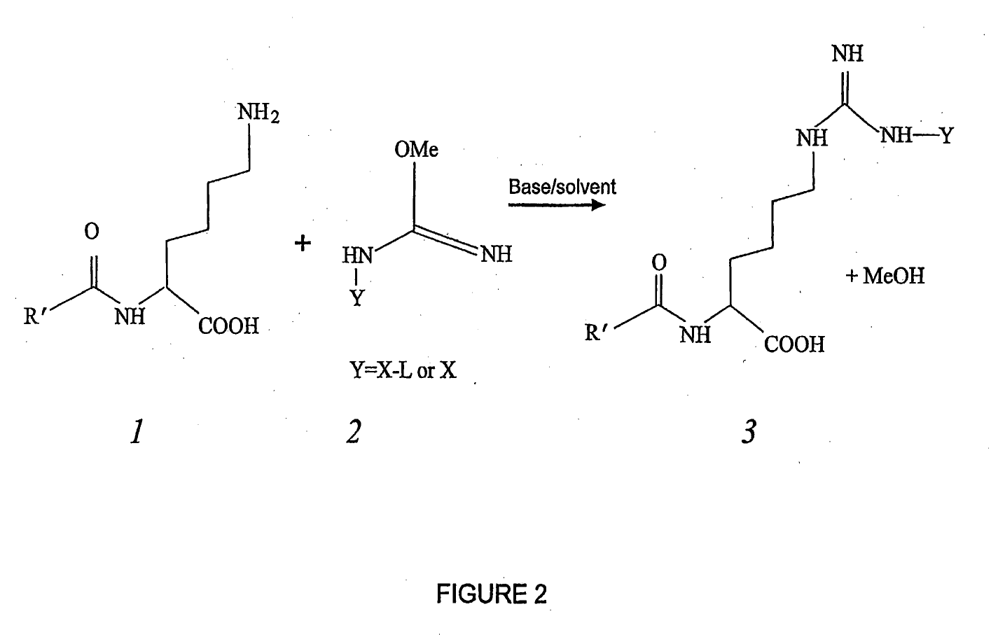

FIG. 2 shows the guanidination of a lysine containing macromolecule into a homoarginine derivative using a binding reagent;

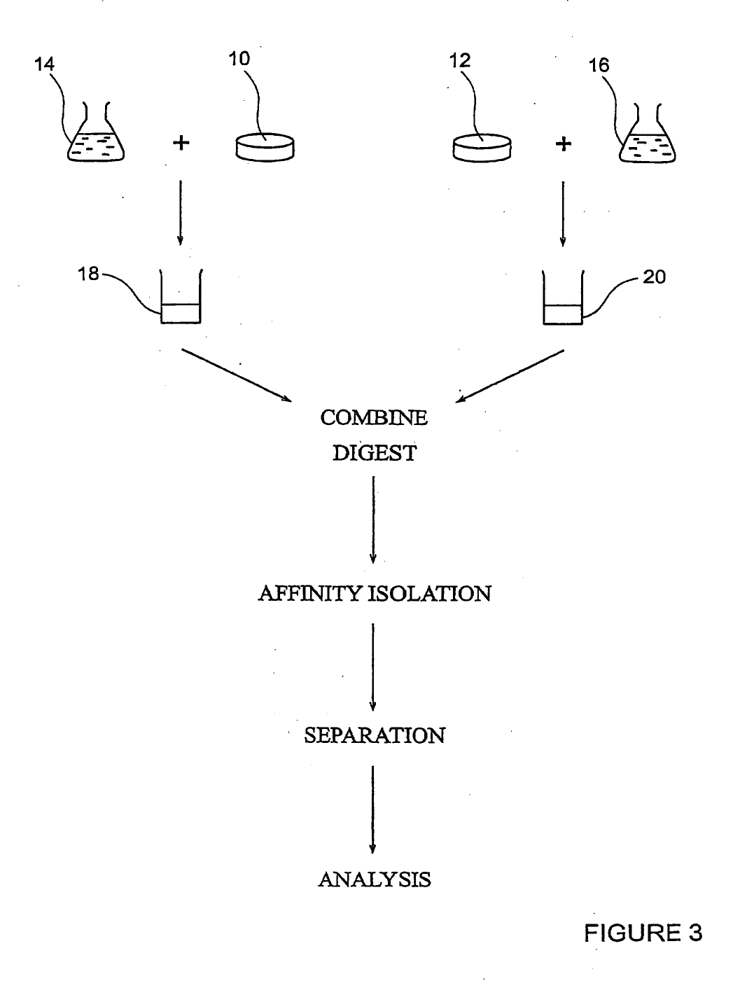

FIG. 3 shows a scheme for quantifying differential protein expression;

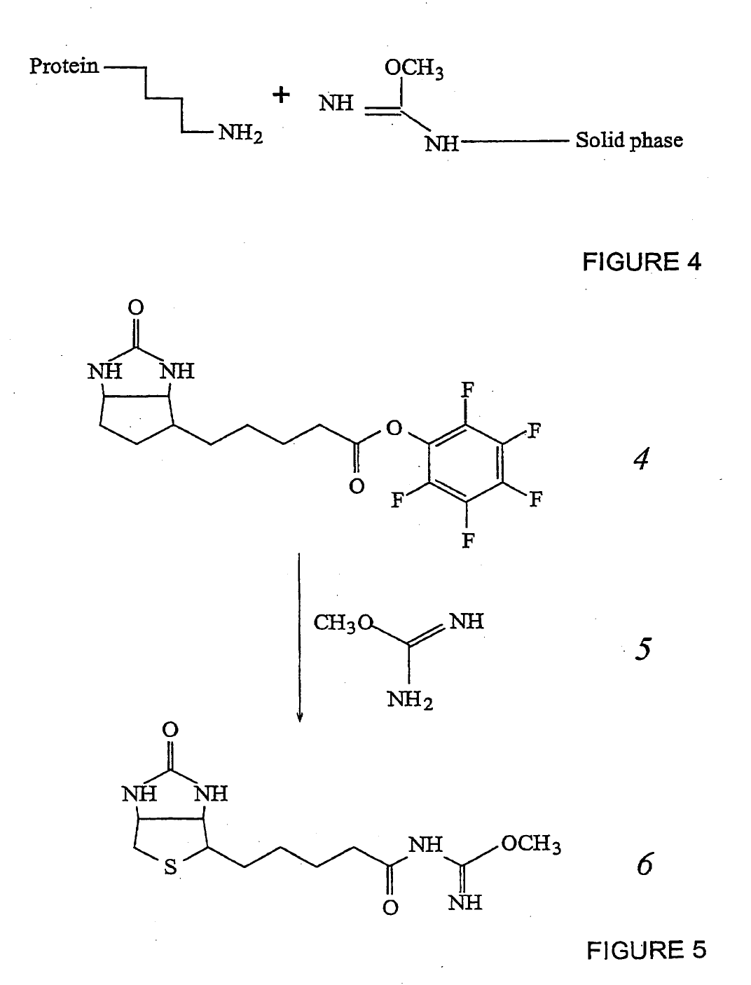

FIG. 4 shows an alternative form of a binding reagent;

FIG. 5 shows a reaction scheme for preparing a binding reagent;

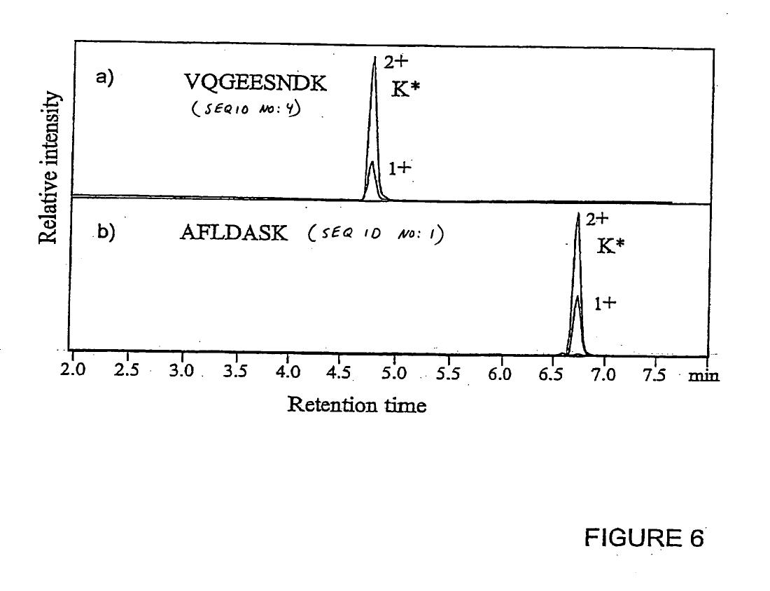

FIG. 6 shows a LC/ES-MS analysis of lysine terminal standard peptides treated with O-methyl isourea. Selected ion monitoring (SIM) of four marker-ions is performed selecting the singly and doubly charged ions of lysine (K) and its corresponding homoarginine terminal peptide (K*), (a) 35 pmole of interleukin [M+2H]²⁺ and [M+H]⁺ at 503.2 and 1005.4 are indicated with ²⁺ and 1+respectively. (b) 50 pmole of AFLDASK [M+2HF]²⁺ and [M+H]⁺ at 376.25 and 751.25 are indicated with 2+ and 1+ respectively. The arrows indicate the expected retention times of the unmodified lysine containing peptides;

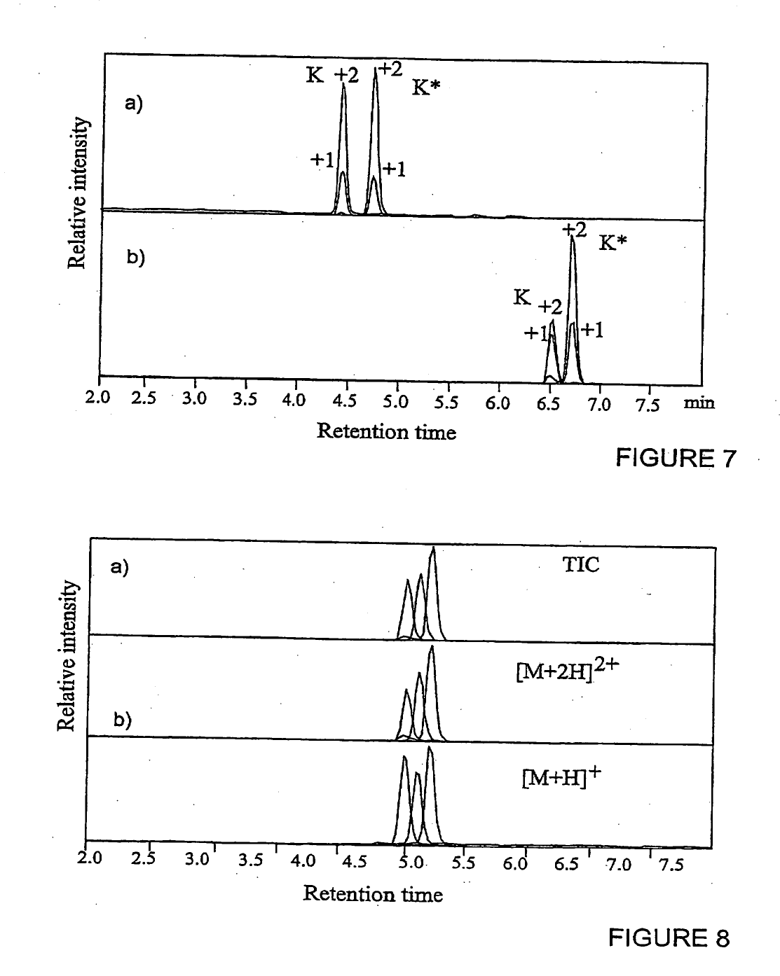

FIG. 7 shows a LC/ES-MS analysis of an equimolar mixture of interleukin (K, 12.5 pmole) with its guanidinated counterpart (K*,12.5 pmole) acquired in selected ionmonitoring. Doubly charged ion signals are indicated with +2 while singly charged ion signals correspond to +1, (b) SIM LC/ES-MS analysis of a equimolar mixture of 20 pmole of AFLDASK with AFLDASK*;

FIG. 8 shows a LC/ES-MS analysis of a solution containing equal amount (15 pmole) of ALFDASK, AFLDASR and the homoarginine terminal analogue AFLDASK*. The lysine terminal peptide is eluted first followed by the arginine terminal counterpart. SIM analysis of all six ions indicates that the homoarginine terminal peptide ions are the dominant signals in the MS chromatogram regardless of the charge state selected;

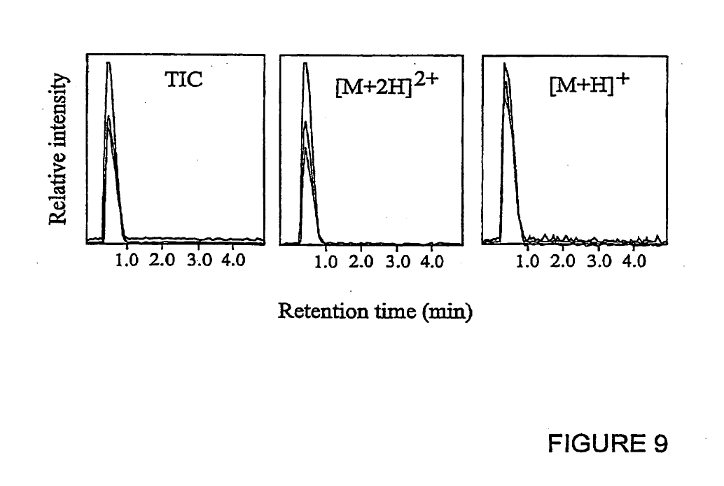

FIG. 9 shows an isocratic separation of a solution containing equal amount (31.5 pmole) of AFLDASK, AFLDASR and the AFLDASK*. The conditions employed correspond to the concentration of mobile phase (0.05% (v/v) TFA and 32.6% (v/v) acetonitrile) at which the peptides were eluted in gradient mode;

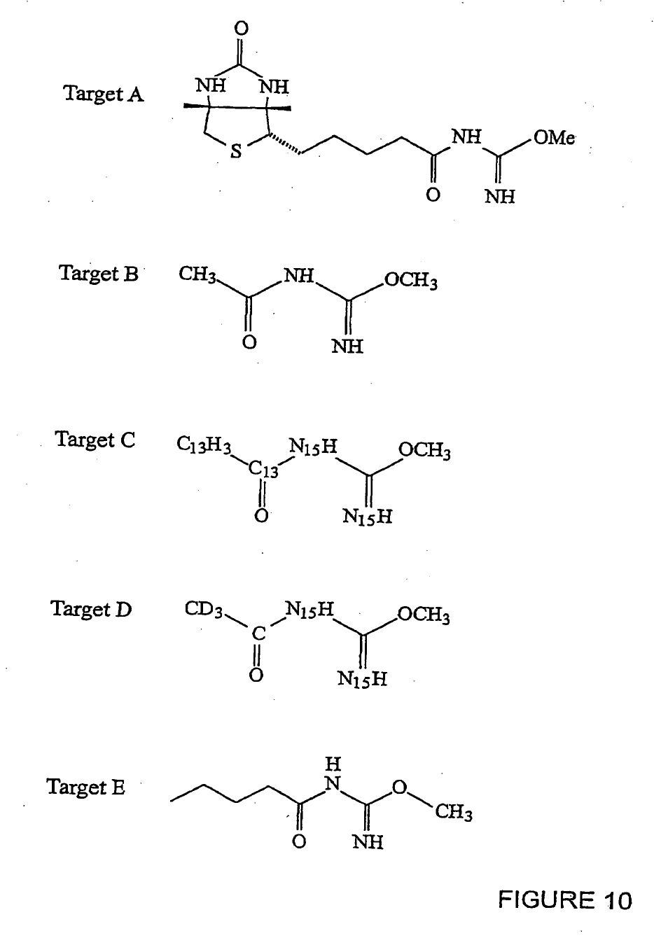

FIG. 10 shows a series of target modules;

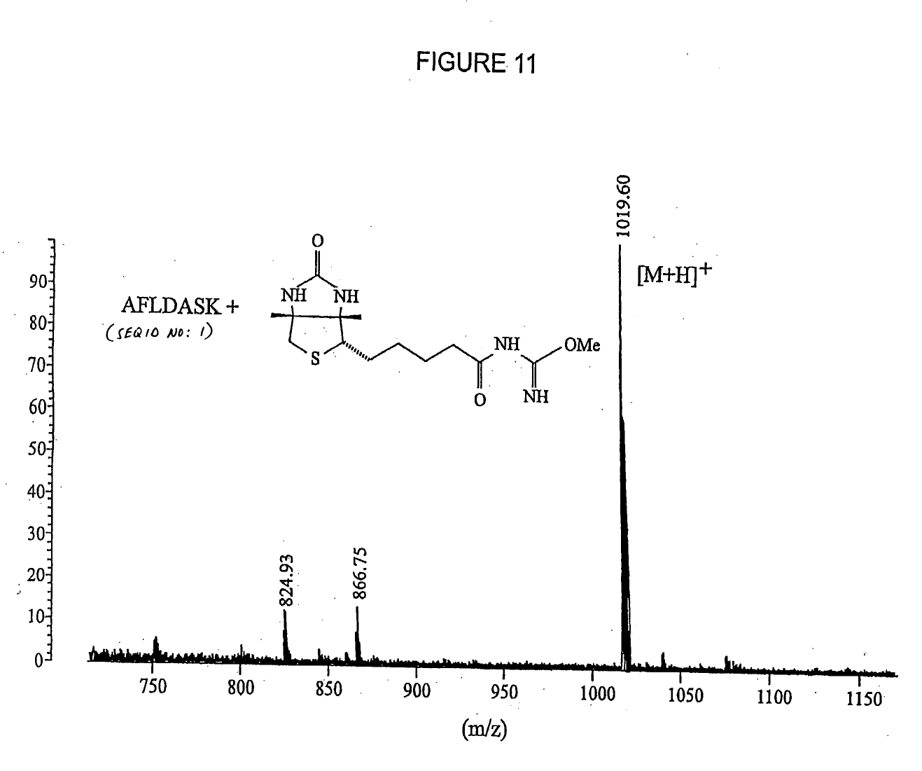

FIG. 11 shows the derivatisation of AFLDASK with target A. The ion signal [M+H] + at m/z 1019 corresponds to the mono-adduct produced by the reaction between target A and the lysine terminal peptide AFLDASK;

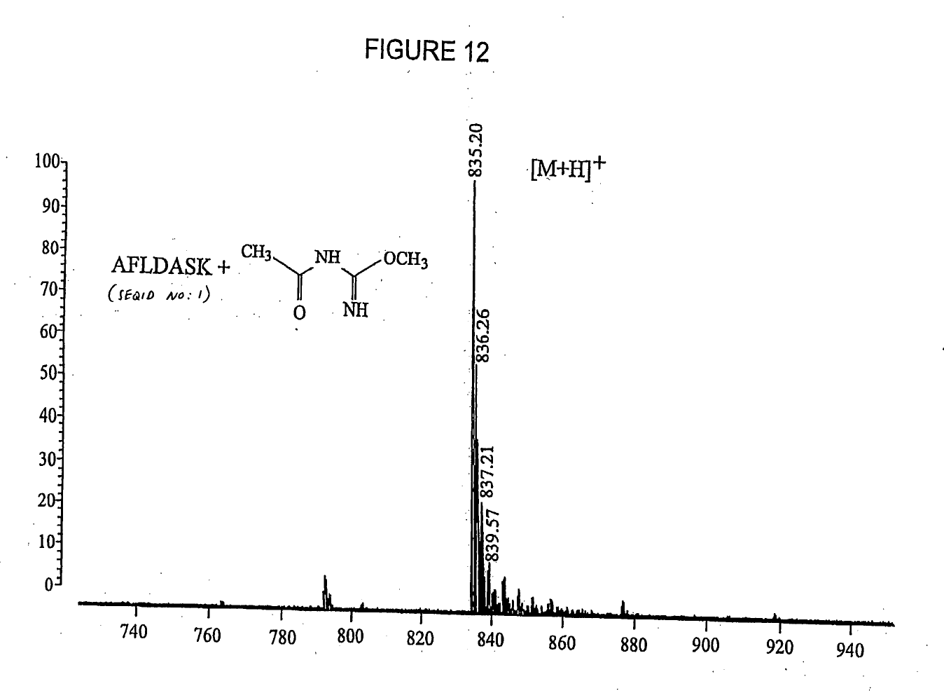

FIG. 12 shows the derivatisation of AFLDASK with 1 -acetyl-2-methyl isourea The ion at m/z 835.2 is the mono-adduct produced by coupling between AMIU and the lysine terminal peptide;

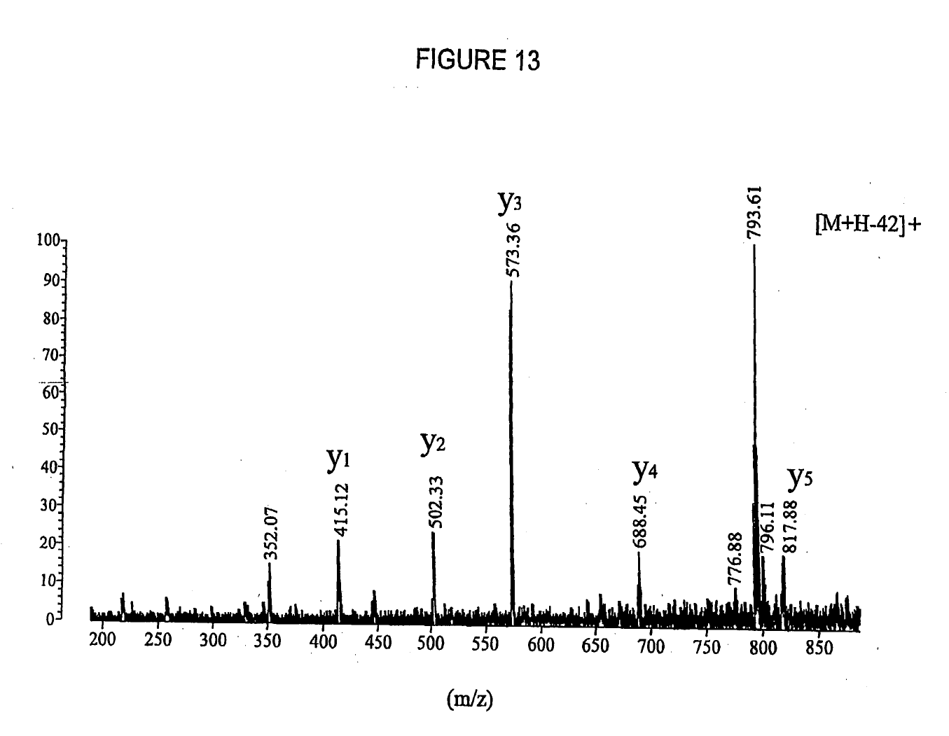

FIG. 13 shows a PSD MALDI TOF spectrum of the ion corresponding the peptide derivatised with target A;

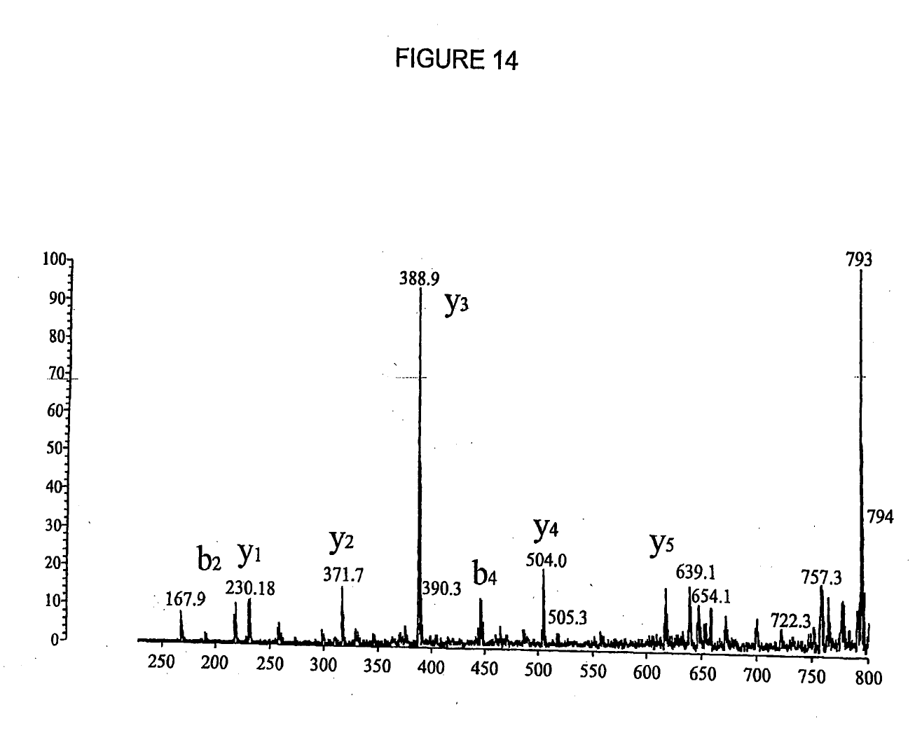

FIG. 14 shows a PSD MALDI TOF spectrum of the AFLDASK peptide derivatised with target B (AMIU);

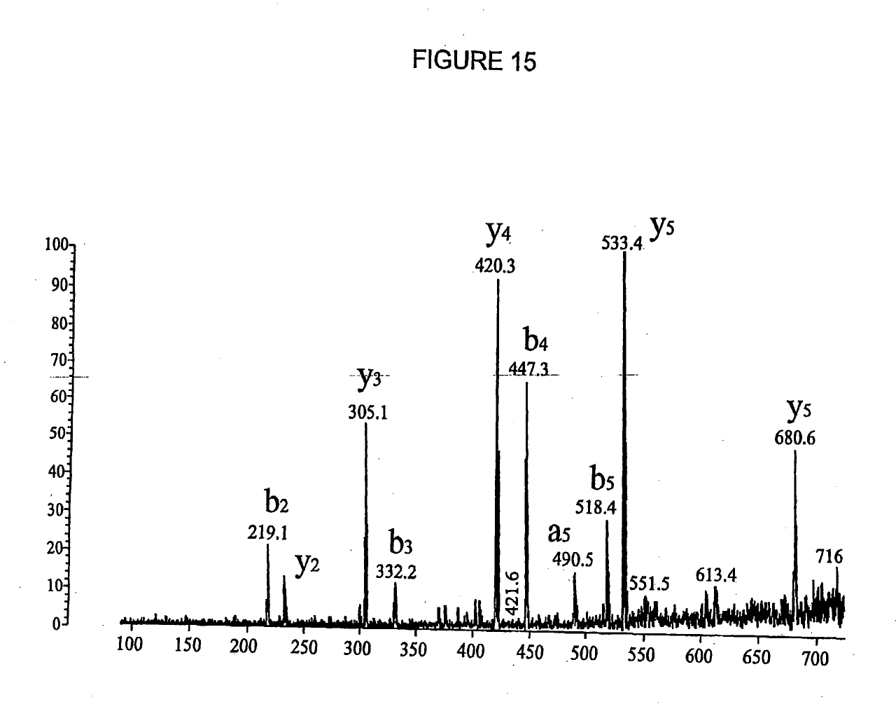

FIG. 15 shows a PSD MALDI TOF spectrum of the AFLDASK; and

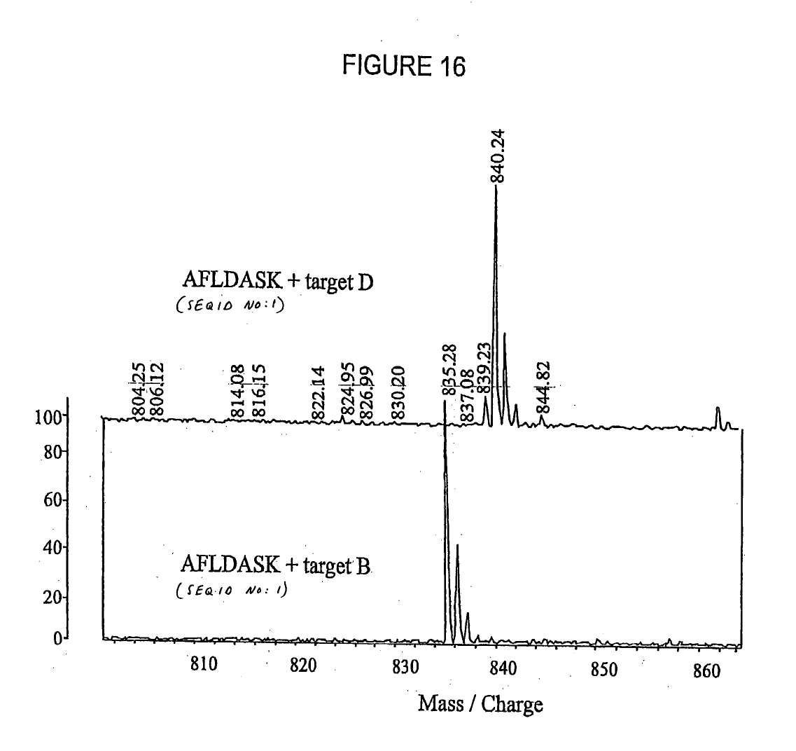

FIG. 16 shows a MALDI spectra of AFLDASK differentially labelled.

The present invention exploits the guanidination reaction to inter alia provide improvements to the technique of Gygi et al and WO 00/11208.

FIG. 1 depicts the guanidination of lysine, typically at around pH 10 or greater, into homoarginine using O-methyl isourea. FIG. 2 depicts a modified scheme in accordance with the invention in which guandination of a macromolecule 1 having a lysine functionality is performed using an isourea derivative 2, producing a homoarginine derivative 3. The macromolecule 1 can be, for example, a peptide or a protein. The isourea derivative 2 is a binding reagent provided by the present invention in which X is an affinity label that selectively binds to a capture reagent. X may be a moiety that can incorporate different isotopic variants. It is possible to utilise an isourea derivative 2 in which a linker moiety L links the affinity label X to the O-methyl isourea moiety.

In a general sense, the invention provides methods and reagents for selectively binding and capturing macromolecules having a lysine functionality. Once bound and captured a macromolecule can be analysed by a suitable technique such as mass spectrometry or a spectroscopic method. In important embodiments the invention provides analytical reagents and analytical methods for the isolation, purification and identification of proteins and peptides in mixtures of proteins and peptides. The method employs binding reagents which exploit the reactivity between lysine amino groups and isourea derivatives to isolate, purify and determine peptides and proteins present in a mixture. Homoarginine derivatives are created which isolate lysine containing proteins and peptides.

The chemistry between a lysine side chain amino group and isourea moiety is well established. In the majority of cases, the presence of the N-terminal amino group does not interfere with the reaction occurring on the lysine amino groups.

An additional advantage associated with the use of guanidination in peptide sequencing stems from the predictable addition of a guanidino moiety into the peptide structure. Selective modification of the E-lysine amino group produces a shift of 42 Da which affects solely ions with the proton located at C-terminus (x,y,z). Comparison of the ion signals deriving from the two product ion spectra enables assessment of the type of ion encountered.

FIG. 3 shows a scheme for quantifying differential protein expression. Two protein mixtures 10, 12 which represent different cell states are treated with chemically identical, isotopically different binding reagents 14, 16. Thus, a first protein mixture 10 may be treated with an isotopically “light” reagent 14, and a second protein mixture 12 may be treated with an isotopically “heavy” reagent 16. Guandination produces a mixture of homoarginine derivatives 18, which, by virtue of the binding reagents utilised, is “light” and a mixture of homoarginine derivatives 20 which, by virtue of the binding reagents utilised, is “heavy”. The mixtures 18, 20 are combined, and proteins (including those forming part of the homoarginine derivatives) are digested to peptides using techniques which are well known in the art. The homoarginine derivatives (now comprising peptides) are isolated using a separation technique commensurate with the nature of the affinity label, such as a chromatographic technique. The isolated homoarginine derivatives are then separated and analysed using a chromatographic technique such as liquid chromatography and mass spectrometry (LC-MS). Micro/nano capillary liquid chromatography (ILC) can be used.

Identical peptides emanating from the mixtures 10, 12 give rise to chemically identical homoarginine derivatives which more or less coelute from the LC. However, due to the “light” and “heavy” nature of the binding reagents, these homoarginine derivatives have different molecular masses which are identifiable in the mass spectrum at different values of mass-to-charge ratio. Thus, relative quantification of protein expression is possible by comparing the relative abundances of the ion peaks corresponding to the “light” and “heavy” homoarginine derivatives. MS/MS analysis of the elutants provides sequence information which enables identification of the protein through computer searching of the experimentally obtained sequence information against databases. Further details of suitable analysis schemes can be found in WO 00/11208 and Gygi et al. It is also possible to perform identification of proteins and peptides using MS/MS without determining relative protein expressions, in which instance isotopic variants of the binding reagents are not required.

Isotopic variants of a binding reagent can be prepared by incorporating different isotopes into the linker moiety, such as is generally described in WO 00/11208 and Gygi et al. One scheme involves deuteration of a hydrogen containing backbone. Additionally, or alternatively, it is possible to utilise ¹⁵N at one or both of the N positions in the isourea moiety.

In place of chromatographic isolation and separation, it may be possible to utilise other capture techniques in order to separate the homoarginine derivatives from the original sample. For example, the affinity label might be attached to a solid surface or a matrix. In a related embodiment, the binding reagent may be linked to a solid support or to a matrix such as shown in FIG. 4. In such a scheme, it is possible to move proteins present in the sample which do not react with the binding reagent in a washing procedure. After attachment to a solid support, proteins can be digested in situ with a proteolytic enzyme and analysed directly, such as by mass spectrometry. The reaction scheme is amenable to automation.

In further embodiments of the invention, calibrations are performed using samples containing known concentrations of proteins, in order to obtain a quantitative relationship between peptide signal obtained during analysis and the absolute amount of protein present in the sample.

Methods

The following synthetic path can be used to synthesise binding reagents.

Preparation of biotin-2,3,5,6-tetrafluorophenyl ester

The method is described at page 43 of WO 00/11208, synthesis number 2 displayed at page 83, using 2 as a residue.

Synthesis and preparation of 0.5M ¹⁴N and ¹⁵N O-methyl isourea Solution

The method described is a modification of a procedure by Burgoyne et al to synthesise a similar compound (Burgoyne, D L, J Org Chem. 2000, 65, 152-155). A solution of 60 mg (1 mmol) ¹⁴N (or ¹⁵N) urea (Aldrich, Milwaukee, Wis.) in 95 μL (126 mg, 1 mmol) of dimethyl sulfate (Aldrich, Milwaukee, Wis.) was heated and agitated at 50° C. for 5 hours. 250 μL of acetone was added and the solution was stirred for 1 hour. 1 mL of diethylether (Aldrich, Milwaukee, Wis.) was then added and the resulting mixture was stirred. The top layer was discarded and the addition of diethylether was twice repeated to afford ˜60 mg (˜100 μL) of O-methyl isourea hemisulfate salt (˜0.5 mmol, 49% yield). The product was dissolved in 900 μL of water to give a ˜0.5M solution.

Reaction Between biotin-2,3,5,6-tetrafluorophenyl ester and O-methyl isourea

The reaction is shown in FIG. 5. Biotin-2,3,5,6-Tetrafluorophenyl ester 4 is reacted with O-methyl isourea 5 to produce a binding reagent 6 through reaction of the primary amine on 5 with the ester moiety on 4.

The biotin containing binding reagent 6 is conveniently used in conjunction with avidin affinity chromatographic techniques.

Synthesis of O-methyl isourea adduct of biotin

A suspension of biotin (244 mg, 1 mmol), O-methyl isourea sulfate (123 mg, 1 mmol) and EDC (384 mg, 2 mmol) in diisopropylethylamine (0.18 ml, 1 mmol) and DMF (19 ml) was stirred overnight. The reaction mixture was then evaporated using a rotary evaporator with a bath temperature of 40° C. and a high-vacuum pump. After evaporation, methanol was added and a solution formed. This material was subjected to a silica gel column chromatography using 10% methanol in DCM as eluent. The product eluted from the column very slowly. A small amount of product was isolated whose ¹H NMR spectrum (in both d4 methanol and d6 DMSO) is consistent with the desired product although some impurity peaks are visible. An attempt was made to further purify this product by recrystallisation from methanol. The product was previously soluble in methanol but upon addition of methanol and warming a portion of the material remained insoluble; the ¹H NMR spectrum of this material in d6 DMSO shows it to be the biotin adduct of the urea (FIG. 10, compound B).

Formation of O-methyl isourea hydrogensulfate

Urea (20 g, 0.33 mol) was added portionwise over one hour to a stirred solution of dimethyl sulfate (30.1 ml, 0.33 mol) at 110-115° C. After stirring for two hours at 110° C., the reaction was cooled to room temperature. Concentrated sulfuric acid (18.5 ml, 0.33 mol) was cautiously dissolved in ether (171 ml) with cooling. This was then added to the oil of the reaction along with acetone (171 ml).

Synthesis of N-acetyl-O-methyl isourea Adduct

A suspension of the O-methyl isourea sulfate (1.23 g, 10 mmol) in acetic anhydride (20 ml) and pyridine (2.4 ml, 30 mmol) was stirred overnight at 30° C. under N₂. The volatiles were then evaporated and methanol was added. A white solid was only partially soluble and was removed by filtration and analysed.

Formation of ¹⁵N₂ labelled O-methyl isourea hydrogensulfate

Urea (¹⁵N₂ labelled) (4 g, 0.064 mol) was added portionwise over 15 minutes to a stirred solution of dimethyl sulfate (5.8 ml, 0.065 mol) at 114-115° C. After stirring for 1.5 hours at 110-115° C., the reaction was cooled to room temperature. Concentrated sulfuric acid (3.6 ml, 0.064 mol) was cautiously dissolved in ether (33 ml) with cooling, and this was then added to the oil of the reaction along with acetone (33 ml).

Formation of ¹⁵N₂ labelled O-methyl isourea sulfate

An opaque solution of barium hydroxide monohydrate (1.94 g, 0.01 mol) in water (10.6 ml) at 60° C. was added to a stirred cooled solution of ¹⁵N₂ labelled O-methyl isourea sulfate (3.52 g, 0.021 mol) in water (˜30 ml) and a milky precipitate was formed. This was centrifuged at 2500 rpm for five minutes and the supernatant was decanted into another centrifuge tube and spun again at 2500 rpm for five minutes to ensure that no solid material was present. The colourless solution was then decanted and evaporated to dryness. Acetone was added and the product crystallised to give the product as a white solid, yield 2.07 g (82%). Melting point 159.0-159.5° C.

Formation of ¹⁵N₂ labelled N-trideuterioacetyl-O-methylisourea

A suspension of ¹⁵N₂ labelled O-methyl isourea sulfate (0.818 g, 6.5 mmol) in acetic anhydride d6 (13.1 ml, 139 mmol) and pyridine (0.53 ml, 6.5 mmol) was stirred overnight at 27° C. The volatiles were then evaporated at T≦30° C. and co-evaporated with toluene. Anhydrous methanol (˜8 ml) was added and the material swirled. A portion of the material did not dissolve and was filtered to give 0.392 g of a white powder which was identified as target compound. Ether was then added to the filtrate and a precipitate formed which was filtered, dried and then washed with anhydrous methanol (˜4 ml) to give a further 0.01 g of pure product. Total yield 0.400 g (51%).

Synthesis of O-methyl isourea Adduct of valeric Acid

A suspension of valeric acid (0.22 ml, 2 mmol), O-methyl isourea sulfate (246 g, 2 mmol) and EDC (0.77 g, 4 mmol) in diisopropylethylamine (0.36 ml, 24 mmol) and DMF (20 ml) was stirred overnight. The reaction mixture was then evaporated using a rotary evaporator with a bath temperature of 35° C. and a high-vacuum pump. After evaporation, the crude product was dissolved in a mixture of DCM, methanol and acetone and added to a silica gel column containing DCM. The desired product eluted in the first three fractions and was fairly pure (as demonstrated by nmr spectroscopy). An attempt was made to re-purify this material by column chromatography (eluent=DCM/methanol) but no material corresponding to the product was isolated.

The reaction was repeated on a larger scale: a suspension of valeric acid (0.44 ml, 4 mmol), O-methyl isourea sulfate (492 mg, 4 mmol) and EDC (1.53 g, 8 mmol) in diisopropylethylamine (0.72 ml, 4 mmol) and DMF (150 ml) was stirred overnight. The reaction mixture was then evaporated using a rotary evaporator with a bath temperature of 35° C. and a high-vacuum pump. After evaporation, dioxane was added and the reaction mixture was filtered, thus removing unreacted O-methyl isourea sulfate. The dioxane soluble material was purified by silica gel column chromatography using dioxane as eluent. The product was obtained as an off-white solid 0.27 g (42%) and was not as pure as the material previously isolated.

### EXAMPLE 1

During analysis of peptide mixtures, many factors influence electrospray response. Selective suppression of ion signals hampers quantification of all components observed in the spectrum. An investigation was performed into the effect of the C-terminal amino acid on electrospray ionisation (ESI) response of peptides. More specifically, the ionisation efficiency of lysine, arginine and homoarginine terminal peptides and the effect of limited modification in peptide structure on differences in ESI response were investigated.

Methods

Interleukin, VQGEESNDK (Sigma), AFLDASR and AFLDASK with purity >96% (New England Peptide Inc) were used in concentrations of 2-20 pmol/μl. All experiments were performed on a single quadruple mass spectrometer LCMS-2010 (Shimadzu, Japan) fitted with an electrospray source. Source conditions were maintained constant during the analysis. Data was acquired in selected ion monitoring mode (SIM) for a total of twelve selected ions. Components were separated on a Phenomenex Luna C₁₈ column (2.0 mm id×50 mm). In gradient mode, mobile phase composition was 0.05% TFA (v/v) for the aqueous phase and acetonitrile/water (9:1 v/v) incorporating 0.05% TFA (v/v) for the organic phase. LC10ADV_{P} HPLC pumps (Shimadzu, Japan) were used to deliver solvent at a flow rate of 250 μl/min.

### RESULTS

Completeness of guandination Reaction.

In order to verify the absence of starting material, lysine containing peptides treated with methyl isourea were loaded onto a C₁₈ column and subjected to liquid chromatography prior to ES analysis. The separation has two advantages: first, it removes the excess of O-methyl isourea which interferes negatively with the ionisation process. Second, all components in the reaction mixture are resolved and eluted separately into the mass spectrometer. Subsequent detection by ES/MS allows comparison and quantification of the relative intensities of lysine and its homoarginine terminal counterpart. FIG. 6 illustrates LC/ES-MS analysis of lysine terminal standard peptides treated with methyl isourea. Selected ion monitoring (SIM) on four specific marker-ions corresponding to the singly and doubly charged ions of lysine and homarginine terminal peptides was used to monitor whether peaks in the total ion chromatogram contain lysine terminal peptides.

These results demonstrate that the guanidation procedure is quantitative.

LC-MS analysis of mixtures containing lysine and arginine terminal peptides.

The completeness of guandination allows preparation of homoarginine terminal peptide solutions with known concentrations. Binary mixtures containing the same quantity of lysine terminal peptide and its analogous homoarginine terminal counterpart were prepared and a comparison between the two ion signals is performed. FIG. 7a shows an MS chromatogram of 20 pmole of interleukin with its guanidinated analogue during LC separation followed by ES. The conditions employed during the chromatographic separation (composition of mobile phases, gradient profile, etc) were kept identical to those used in the previous experiment (see FIG. 6). Total ion chromatogram (TIC) traces of homoarginine terminal peptides (corresponding to the sum of the singly and doubly charged ion signals) have higher relative intensity than lysine terminal ones. The main contribution is due to the relative ES response of the doubly charged ion peaks. This observation is also made with an equimolar mixture of AFLDASK with its guanidinated counterpart AFLDASK* (FIG. 7B).

LS-MS analysis of a mixture containing AFLDASK, AFLDASR and its homoarginine terminal counterpart AFLDASK*.

To investigate how minor variations in peptide structure may affect signal intensity, the arginine terminal analogue AFLDASR was added to the mixture. FIG. 8 indicates the ion chromatograms of a solution containing 31.5 pmole each of AFLDASK, AFLDASR and the homoarginine terminal derivative AFLDASK* separated by LC prior to MS detection. The arginine terminal peptide displays an ion chromatogram with intensity higher than the corresponding AFLDASK in accordance with the higher basicity of the arginine. By comparing the two sets of peptide ions produced by AFLDASR and AFLDASK*, the latter one generally displays ion signals with higher intensity. Unlike the lysine and arginine/homoarginine terminal peptides (AFLDASK vs AFLDASR/AFLDASK*) where the difference in ionisation efficiency can be related to the higher stabilisation of the protenated peptide, in the case of the AFLDASK and AFLDASK* the difference in ES response cannot be simply related to the effect of the guanidino group as both are identical. The difference can be attributed to the increased level of proton solvation provided by the extra methylene group in the peptide backbone. Effect of isocratic separation on ESI response of AFLDASK, AFLDASR and AFLDASK* peptide ions.

The effect of co-elution of three peptides on their ESI response was studied. The mixture previously analysed (see FIG. 8) was run isocratically. The mobile phase was prepared with the same composition used to elute AFLDASR, AFLDASK and AFLDASK* in the gradient mode. Hence, no separation is achieved and all three peptide elute simultaneously into the mass spectrometer. None of the ion signals in the MS chromatogram are suppressed compared with the ion signals observed in the LC-MS experiment (see FIG. 8). The co-presence of three peptides different in gas/phase basicity shows no effect on their ESI response. FIG. 9 displays MS chromatograms of AFLDASK, AFLDASR and AFLDASK* for singly and doubly charged protonated ions.

### CONCLUSIONS

The completeness of the conversion of lysine into homoarginine enables quantitative evaluation of how C-terminus modification affects ESI response. Guanidation produces variation in either gas phase basicity (between lysine and homoarginine) or in peptide structure (between arginine and homoarginine) resulting in an enhanced signal intensity for the guanidinated peptide. Co-elution and concomitant ionisation of all three peptides shows no effect on the relative ESI responses.

### EXAMPLE 2

Target Molecules

FIG. 10 shows the structures of the biotin derivative (Target A), 1-acetyl-2-methyl isourea (AMIU) (Target B), and the heavy isotopic variant of 1-acetyl-2-methyl isourea containing N¹⁵ and C¹³ (Target C). FIG. 10 also shows Target D which is a heavy isotopic variant of 1-acetyl-2 methyl isourea incorporating deuterium instead of C¹³, and a valeric acid derivative (Target E).

Reactivity Between Targets (A, B) and lysine Terminal peptide

The reactions between the newly synthesised compounds, A,B and a standard peptide (AFLDASK) were tested by using 100 pmole of peptide. Experiments were performed to assess the reactivity of Target A (FIG. 11) and Target B (FIG. 12) with lysine terminal peptides. MALDI analysis was used to evaluate the purity of these compounds. Only one ion signal corresponding to the derivatised peptide is present in the MALDI spectrum. From the analysis performed, it can be assumed that the reaction is quantitative and no starting material is left in the reaction mixture. Additionally, the presence of just one main ion peak in each spectrum rules out the possibility of the multiple derivatisation due to the amino group at N-terminus.

MS/MS Analysis of the Derivatised AFLDASK

Since the N-terminal amino group could interfere with the reaction, tandem mass spectrometric analysis was conducted on the derivatised ions. The product ion spectra were found to demonstrate that the y ions are affected by a shift due to the reaction occurring between the lysine amino group and the reagent. No evidence of coupling on N-terminus was found. MS/MS analysis was performed by exploiting the natural decomposition of the ions in the free-field region of the time-of-flight instruments (PSD). FIG. 13 shows the MS/MS spectrum of the derivatised AFLDASK with Target A. y ions dominate the spectrum and are shifted by 268 amu. Similarly the product ion spectrum of the reaction between AFLDASK and AMIU illustrates a similar trend (FIG. 14). FIG. 15 illustrates the fragmentation pattern of the underivatised AFLDASK.

MALDI spectra of Differentially Labelled lysine terminal peptides

FIG. 16 displays the spectra of AFLDASK labelled with Target B (lower spectrum) and Target D (upper spectrum). As expected the difference in mass between the two peptide ions is 5 Daltons.

1. A method for selectively binding micromolecules having a lysine functionality comprising the steps of:
providing a sample containing one or more species of macromolecules, each having a lysine functionality;
providing a binding reagent having the formula
X—NH—C(═NH)—OR
or
X—L—NH—C(═NH)—OR
where X is an affinity label that selectively binds to a capture reagent, R is a residue group, and L is a linker moiety;
introducing the binding reagent to the sample so as to effect a guanidination reaction between the binding reagent and said one or more species of macromolecules, thereby producing one or more affinity label containing homoarginine derivatives;
optionally modifying the affinity label containing homoarginine derivatives to produce further affinity label containing homoarginine derivatives; and
capturing affinity label containing homoarginine derivatives using the capture reagent that selectively binds X.

2. A method for analysing one or more proteins, protein functions and/or peptides in one or more samples comprising the steps of:
providing a binding reagent having the formula
X—NH—C(═NH)—OR
or
X—L—NH—C(═NH)—OR
where X is an affinity label that selectively binds to a capture reagent, R is a residue group, and L is a linker moiety;
introducing the binding reagent to the one or more samples so
as to effect a guanidination reaction between the binding reagent and proteins and/or peptides having a lysine functionality, thereby producing one or more affinity label containing homoarginine derivatives;
optionally modifying the affinity label containing homoarginine derivatives to produce further affinity label containing homoarginine derivatives;
capturing affinity label containing homoarginine derivatives using the capture reagent that selectively binds X; and
performing an analysis of affinity label containing homoarginine derivatives.

3. A method according to claim 2, in which the step of modifying the homoarginine derivatives comprises converting proteins present into peptides.

4. A method according to claim 2, in which the proteins, protein functions and/or peptides are identified by the analysis of the affinity label containing homoarginine derivatives.

5. A method according to claim 4, in which the analysis comprises the step of comparing data generated by an analytical technique with sequence databases.

6. A method according to claim 2, in which relative expression levels of proteins in two or more samples containing proteins are determined comprising the steps of:
providing a series of binding reagents having the formula
X—NH—C(═NH)—OR
or
X—L—NH—C(═NH)—OR
where X is an affinity label that selectively binds to a capture reagent, R is a residue group, and L is a linker moiety, and wherein the chemical formulae of the binding reagents in the series are identical but each binding reagent in the series comprises a different combination of isotopes so that binding reagents in the series are isotopically labelled by way of the molecular mass of each binding reagent in the series being different to the molecular masses of the other binding reagents in the series;
introducing a different binding reagent from the series to each sample so as to effect, in each sample, a guardination reaction between a binding reagent and moieties having a lysine functionality, thereby producing a plurality of isotopically labelled, affinity label containing homoarginine derivatives;
combining the samples;
optionally converting proteins into peptides; capturing affinity label containing homoarginine derivatives using the capture reagent that selectively binds X; and
performing an analysis of affinity label containing homoarginine derivatives in which the relative abundances of a subset of homoarginine derivatives which differ only by virtue of their isotopic labelling are measured, thereby determining the relative expression levels of the protein from which the subset of homoarginine derivatives originated.

7. A method according to claim 6 in which the step of converting proteins into peptides comprises converting proteins present in the affinity label containing homoarginine derivatives into peptides.

8. A method according to claim 6, in which proteins, protein function and/or peptides are identified by the analysis of the affinity label containing homoarginine derivatives.

9. A method according to claim 8, in which the analysis comprises the step of comparing data generated by an analytical technique with sequence data.

10. A method according to claim 2, in which the analysis comprises mass spectrometric analysis.

11. A method according to claim 10, in which the mass spectrometric analysis comprises tandem mass spectrometry.

12. A method according to claim 2, further comprising the step of releasing captured affinity label containing homoarginine derivatives from the capture reagent prior to the step of performing an analysis.

13. A method according to claim 12 in which the capture reagent comprises part of a chromatographic separation system which separates chemically different affinity label containing homoarginine derivatives.

14. A method according to claim 13 in which the chromatographic separation system utilises liquid chromatography.

15. A method according to claim 13 in which the chromatographic separation system utilises gas chromatography.

16. A method according to claim 2 in which absolute quantification of the proteins and/or peptides is obtained.

17. A method according to claim 1 in which R is an alkyl group.

18. A method according to claim 17 in which R is CH.

19. A method according to claim 1 in which X is an alkyl group.

20. A method according to claim 19 in which X is CH₃.

21. A method according to claim 20 in which the binding agent is CH₃CONHC(═NH)OCH₃.

22. A reagent for selectively binding molecules having a lysine functionality having the formula
X—NH—C(═NH)—OR
or
X—L—NH—C(═NH)—OR
where X is an affinity label that selectively binds to a capture reagent, R is a residue group, and L is a linker moiety.

23. A reagent according to claim 22, in which X is biotin or a modified biotin.

24. A reagent according to claim 23 in which X is an alkyl group.

25. A reagent according to claim 24 in which X is CH₃.

26. A reagent according to claim 22, in which R is an alkyl group.

27. A reagent according to claim 26 in which R is CH₃.


SEQUENCE LISTING (4 sequences)

SEQ ID NO: 1 - protein, 7 aa, Artificial Sequence
  Synthetic peptide made by New England Peptide, Inc. Gardner, MA; Lysine terminal standard peptide AFLDASK
AFLDASK

SEQ ID NO: 2 - protein, 7 aa, Artificial Sequence
  Synthetic peptide made by New England Peptide, Inc. Gardner, MA; Homoarginine terminal standard peptide made by guanidination of the terminal Lys and identified in the specification as AFLDASK*
AFLDASR

SEQ ID NO: 3 - protein, 7 aa, Artificial Sequence
  Synthetic peptide made by New England Peptide, Inc. Gardner, MA; Arginine terminal standard peptide AFLDASR
AFLDASR

SEQ ID NO: 4 - protein, 9 aa, Artificial Sequence
  163..171: Interleukin 1 beta, Homo Sapiens; Synthetic peptide made by Sigma, Inc.
VQGEESNDK